(19) United States
(12) Patent Application Publication
(10) Pub. No.: US 2009/0304777 A1
(43) Pub. Date: Dec. 10, 2009

(54) AGENT ADMINISTRATION

(75) Inventor: Rex Munday, Hamilton (NZ)

Correspondence Address: KNOBBE MARTENS OLSON & BEAR LLP, 2040 MAIN STREET, FOURTEENTH FLOOR, IRVINE, CA, 92614, US

(73) Assignee: AG Research Limited, Hamilton (NZ)

(21) Appl. No.: 11/908,708

(22) PCT Filed: Feb. 27, 2006
(86) PCT No.: PCT/NZ06/00031

§ 371 Date: Aug. 5, 2009

(30) Foreign Application Priority Data
Mar. 14, 2005 (NZ) 538813

(51) Int. Cl. A61K 9/22

(52) U.S. Cl. 424/438

(57) ABSTRACT

An intra-ruminal bolus and method of manufacture is described that releases a therapeutically effective amount of at least one beneficial agent to a ruminant animal over a time period of less than approximately 21 days. The bolus includes at least one beneficial agent, at least one densifier material, at least one binding agent, and at least one releasing agent. The bolus is manufactured using a ratio of binding agent to releasing agent that is tailored to achieve the desired rate of release and timing of delivery. This ratio is in the order of approximately 1 part binding agent to between approximately 0.01 and approximately 2 parts releasing agent and may be varied within this range in order to obtain the preferred release length of time. The bolus is particularly useful for delivery of anti-parasitic compounds but may also be used for delivery of other active agents such as trace elements including selenium and cobalt.

### TECHNICAL FIELD

This invention relates to agent administration. In particular this invention relates to a device and associated method of manufacturing the device for administration of agents to animals. More specifically, the invention relates to a device and associated method of manufacturing the device that releases one or more beneficial agents including anti-parasitical agents to an animal at a sustained rate over a defined time period.

### BACKGROUND ART

Delivery of beneficial agents is an important factor in therapy. Achieving delivery of a therapeutic to a human or non-human animal at the desired level and for the desired duration is generally of importance to ensure maximal efficacy and safety.

For the purposes of the discussion below, reference is made to delivery of beneficial agents to treat parasite infestations. However, this should not be seen as limiting, as it should be appreciated by those skilled in the art, that the principles of agent delivery for parasite treatment may also be applied to delivery of other agents.

Parasites are a major production-limiting factor in livestock grazing systems throughout the world, and most production systems rely heavily on the use of anthelmintic drugs to control infestation in livestock. However, in some countries this dependence on the use of anthelmintics is now threatened by the development of resistance amongst parasite populations.

Countries such as South Africa, Australia and countries in parts of South America already have serious resistance problems with parasites of sheep and goats. New Zealand, Great Britain and France also have significant and developing resistance problems. In Australia, for example, almost every sheep farm has resistance to at least one “action-family” of anthelmintic. Survey results indicate that on more than 90% of Australian sheep farms at least two action-families (the benzimidazole and levamisole groups) are less than fully effective due to resistance. Furthermore, the last 2-3 years have seen a rapid increase in the prevalence of resistance to the third action-family, the macrocyclic lactone (ML) group.

By comparison, New Zealand has resistance on about 60% of sheep farms, but most still have effective use of at least two action-families of anthelmintic compound. However, the problem continues to worsen and recent years have seen confirmation of ML resistance in sheep flocks. In addition, New Zealand has an un-quantified but significant problem with ML resistance in parasites of cattle. Current anthelmintic preparations, the reasons for parasite resistance to these preparations, and a practicable and effective method for overcoming this resistance have been discussed in detail in at least the applicants' co-pending application, PCT/NZ2004/000267.

In essence, a preferred method useful for overcoming parasite resistance involves the concurrent administration of two or more anthelmintics, delivered continuously to the animals for a discrete time period of 3 to 14 days via a controlled release device. It should be appreciated that this time period may vary for alternative agents and in the inventors' experience the time period may be up to 21 days. Controlled release devices for delivery of beneficial materials to ruminants are well known to individuals skilled in the art. Many different devices are known, but existing devices have limitations in respect of delivery according to the above preferred method.

One example of a controlled release device is that described in WO 03/004059. This publication describes use of tiny implants or pellets which are injected subcutaneously into the animal to be treated, such as a dog. Other methods of administration are not exemplified or even considered. As injections are not always a preferable method of administration, particularly when treating large numbers of animals, the device and method described in WO 03/004059 is not ideal.

Other controlled release devices consist of an apparatus that is retained in the rumen of a ruminant animal, and which releases a therapeutic dose over a period of time. Such intra-ruminal devices involve suspension of the active ingredients in an aqueous environment. This is not desirable as, under these conditions, the different actives will interact resulting in mutual decomposition and loss of efficacy. A dry, solid matrix for holding the actives for delivery is preferable.

Other intra-ruminal controlled release devices contain plastic or metal components which remain in the animal's rumen after dispersion of the active component. Obviously there is a limit to the number of expired devices that can remain in an animal without adverse consequences for the animal, and therefore there is a limit to how many of these devices can be given to an animal over its lifetime. Furthermore, the component residues pose problems in freezing works when offal is processed.

Many intra-ruminal controlled release devices depend upon density for retention in the rumen. One type of bolus has discrete solid metal weights at one end. These remain in the rumen after the payload of the device has been delivered, and with multiple treatments of an animal, many such pieces of metal may be present within the rumen when the animal comes to slaughter. Such metal pieces cause severe damage to the cutting equipment used in the preparation of offal by-products in processing facilities.

In an attempt to ameliorate this problem, the use of sintered metal blocks has been proposed, which shatter during processing and which are claimed to not damage machinery. However, sintered metal is still undesirable, as the metal fragments may pass into the by-products and become a contaminant source. The presence of metal fragments in, for example, pet-food, is unacceptable. A device that leaves no metal or plastic residues after its load of active has been released is required.

A further problem of existing controlled release devices is that their actives are released at a relatively low rate over a period of two or more weeks or even several months. As mentioned above, a release period over 3 to 21 days is preferable for ideal parasite treatment and prevention of resistance.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### DISCLOSURE OF INVENTION

The invention broadly relates to a bolus designed to release one or more beneficial agents over a time period of less than approximately 21 days and at a therapeutically effective dose level.

According to one aspect of the present invention there is provided an intra-ruminal bolus for release of a therapeutically effective amount of at least one beneficial agent to a ruminant animal over a time period of less than approximately 21 days that includes:
(a) at least one beneficial agent;
(b) at least one densifier material;
(c) at least one binding agent;
(d) at least one releasing agent; and,
   characterised in that the ratio of binding agent and releasing agent is tailored to achieve the desired rate of release and timing of delivery.

For the purposes of this specification, the term ‘bolus’ refers to a large pill type composition that is administered orally and which tends to stay and erode or dissolve in the rumen of the animal over a prolonged time period.

The inventors have unexpectedly found that by careful manipulation of the components used to manufacture the bolus, it is possible to produce a bolus that delivers a beneficial agent or agents at ideal rates and over ideal time periods. Without consideration of these critical factors, a bolus is likely to be produced that either does not release enough beneficial agent, releases too much agent, and/or releases the agent or agents at variable rates. Also, in the case of parasites, the targeted organism may also develop resistance to the agent or agents if the dose is insufficient to kill the organism.

A particularly critical factor established by the inventors is the ratio of binding agent to releasing agent. It is the inventors' understanding that the bolus erodes away due to the releasing agent dissolving in fluids present in the rumen. As the releasing agent dissolves, it forms an emulsion with the binding agent which, through the mixing action of the rumen fluid is washed away along with beneficial agent and densifier.

To a lesser extent, the densifier and beneficial agent materials may also influence the rate of release. As a result, the ratio of binding agent to releasing agent must also account for the densifier and beneficial agents chosen.

In preferred embodiments, the ratio of binding agent to releasing agent ranges from approximately 1 part binding agent to between approximately 0.01 and approximately 2 parts releasing agent to achieve a sustained release rate over a time period of less than approximately 21 days. In one preferred embodiment, the ratio is approximately 1:1 to achieve release duration of approximately 7 days. It should be appreciated that the ratio may be varied through simple trial and error to achieve alternative rates of release and alternative time periods for release.

Preferably, the beneficial agent includes at least one anthelmintic compound. Most preferably, the composition includes two anthelmintic compounds from differing action-families of anti-parasitic activity. In one embodiment, the anthelmintic compounds are albendazole and abamectin. However, this should not be seen as limiting, since the device may equally be used for delivery of other materials with or without anti-parasitic agents, such as mineral or nutritional supplements, anti-bacterial agents, anti-viral agents, anti-fungal agents, or other substances beneficial to the health and/or productivity of the ruminant animal. For example, in one embodiment envisaged by the inventors, a source of selenium and/or cobalt is added to the bolus. This is done to rectify deficiencies or avoid deficiencies of these elements in the animals.

Preferably, the densifier material forms the main component of the central part of the bolus, or the ‘core’, and is characterised by having a density sufficient to retain the bolus within the animal rumen and prevent regurgitation. More preferably, the density of the bolus as a whole is greater than 1.5 g/cc, primarily due to the densifier content. More preferably, the bolus density is at least 2.4 g/cc where the ruminant animal is an adult sheep. It should be appreciated that the exact density will vary depending on the animal species and animal size to which the bolus is administered. In preferred embodiments, the densifier material is envisaged as including one or more materials selected from: iron, zinc, tungsten, cement or similar density materials.

Most preferably, the densifier material may be iron in a powder form. Iron is advantageous as it has a high density which results in only a small amount of iron being necessary to achieve the desired density sufficient to retain the bolus in the animal. It is also particularly advantageous for delivery of anthelmintic compositions. Some nematode worm parasites live on blood, and cause anaemia in parasitized animals. Iron in the densifier may transfer to the animal blood stream, and therefore facilitate compensatory erythropoiesis after the worms have been destroyed, accelerating the animal's return to full health.

In an alternative embodiment, iron powder may be substituted by copper metal (in the form of powder or needles) or copper salts. Copper is also advantageous as an adjuvant as it is a beneficial element to an animal and often is administrated as a supplement. Furthermore, there is evidence that copper itself is parasiticidal, and therefore inclusion of this metal will add to the parasiticidal effect of the composition.

For the purposes of this specification, the term ‘binding agent’ refers to one or more compounds which may be used to retain the bolus as substantially one mass for the purposes of bolus manufacture.

Preferably, the binding agent or agents are hydrophobic. More preferably, the binding agent or agents may include: one or more fats, waxes, gums, fatty acids, fatty acid esters, fatty acid amides, fatty acid alcohols or derivatives thereof, glycerol esters and similar physiologically acceptable organic compounds with equivalent characteristics being that they are physiologically acceptable and are able to bind particulate material.

Preferably, the releasing agent or agents are characterised by being hydrophilic and having surfactant properties. More preferably, the releasing agent or agents may include: one or more detergents, soaps, fatty acid salts, polyoxyethylene alcohols and derivatives, polyethylene glycol and derivatives thereof, and like physiologically-acceptable surface-active agents.

In preferred embodiments, binding and releasing agents have a melting point which is higher than the internal temperature of the animal. This is to ensure that the agents do not melt within the animal after ingestion and inadvertently alter the rate of release. However, this should not be seen as limiting as it should be appreciated that melting of the materials may be an alternative or even complementary method of releasing the beneficial agent or agents.

Preferably, the above composition erodes away and leaves no residue after the beneficial agent or agents have been released. This has the advantage of not leaving residues in the animal that may interfere with equipment used to process the animal, as is the case with some existing controlled release devices. In addition, the composition does not pose a contamination risk that devices which use sintered metals do.

Preferably, the bolus releases beneficial agent at a sustained rate during the course of treatment. Most preferably, the amount of agent released is equivalent to that which would be administered if the animal were to have a single oral dose drench administered on a daily basis. It should be appreciated by those skilled in the art that a single oral dose or drench delivers a dose of beneficial agent that decays away quickly. Treatment with the composition of the present invention provides longer duration of exposure to the beneficial agent than a single oral dose or drench.

Preferably, beneficial agent is released over a time period of approximately 3 to 21 days. More preferably, the beneficial agent or agents are released over a time period of approximately 7 days. This has been found by the inventor as being a critical time period to both remove parasite infection and avoid parasites developing resistance to the anti-parasitic agent.

Preferably, the ruminant animal to which the device is administered is a sheep or cow.

Preferably, the bolus is coated with a first and second layer of coating agent.

For the purposes of this specification the term ‘sealing agent’ refers to a material that closes up the minute pores in the bolus, thereby improving the adhesion of the second coating agent.

Preferably, the first layer coating is formed from at least one sealing agent, applied to the whole bolus except the distal planar end. The sealing agent includes: natural or synthetic resins, such as shellac, or synthetic polymers, such as polyurethane. Preferably, the first layer is formed from a 5 to 20% w/v solution of shellac in absolute alcohol.

Preferably, the second layer is applied to the exterior of the first layer coating. Preferably, the second layer is formed from: a wax, a mixture of waxes or a wax or waxes combined with an inert material or a synthetic polymer that is substantially impervious to rumen fluid.

Preferably, the bolus may have a density greater than approximately 1.5 g/cc. More preferably, the density is 2.5 g/cc.

Preferably, the bolus size is greater than approximately 10 mm in diameter. It should be appreciated that the preferred bolus size may be dependent on the animal size to which the bolus is administered. Preferably, the bolus is sized to be sufficiently large to prevent passage from the rumen to the reticulum.

In further embodiments, the composition also includes additional materials selected from: fillers, excipients, modifiers, humectants, stabilisers, emulsifiers, and other physiologically and pharmaceutically acceptable materials.

According to a further aspect of the present invention there is provided a method of manufacturing an intra-ruminal bolus for release of a therapeutically effective amount of at least one beneficial agent to a ruminant animal over a time period of less than approximately 21 days by the steps of:
(a) forming a substantially homogenous mass by melting and mixing together materials selected from:
   i. at least one beneficial agent;
   ii. at least one densifier material;
   iii. at least one binding agent;
   iv. at least one releasing agent; and,
(b) adding the mass to a mould to form a bolus; and,
characterised in that the ratio of binding agent and releasing agent used in step (a) is tailored to achieve the desired rate of release and timing of delivery.

In an alternative embodiment, the mass formed from step (a) is cooled until it solidifies and subsequently ground to a fine powder and formed into a bolus by compression within a die. It should be appreciated by those skilled in the art that the pressure used to compress the powder also influences the desired rate of release. The pressure to use may be determined by a person skilled in the art through routine trial and experimentation.

Preferably, the bolus is shaped in the form of a cylinder or other shape suitable for administration into the rumen of the animal. More preferably, the bolus is shaped so that one distal end of the bolus is substantially hemispherical and the other distal end is substantially planar. It is the inventors' understanding that a planar end ensures the even release of active ingredient as the surface area remains approximately constant as the bolus erodes.

Preferably, all of the materials selected in step (a) (binding agent or agents, releasing agent or agents, densifier(s) and beneficial agent or agents) are melted together before step (b) is completed.

Preferably the mass is added to a mould in step (b) by methods selected from: extrusion, pouring, injection, and combinations thereof.

According to a further aspect of the present invention the binding agent or agents, releasing agent or agents and densifier(s) are melted together, cooled and ground into particles to form a ground material. The beneficial agent or agents are then mixed with the ground material and the bolus then formed in step (b).

Preferably, a further step (c) is completed after step (b) of:
(c) coating the bolus with at least one coating agent.

Preferably, two layers (a first and second layer of coating agent) are used to coat all but the planar distal end of the bolus.

Preferably the first coating layer is formed from a 5 to 20% w/v solution of shellac in absolute alcohol.

Preferably, the second layer is formed from: a wax, a mixture of waxes or a wax or waxes combined with an inert material or a synthetic polymer that is substantially impervious to rumen fluids. More preferably, the second layer is formed from beeswax and carnauba wax.

Preferably, the mixture of step (a) is heated to between approximately 120° C. and approximately 140° C. It should be appreciated however that the temperature used will be determined by the melt temperature of the binding and releasing agents used.

Preferably, before step (b) and after step (a), the mixture from step (a) is ground to a fine powder.

It should be appreciated from the above description that there is provided a composition formulated for sustained release of an active agent and which addresses problems of prior art methods including low and/or fast decay release rates and unwanted residues left in the animal post release of the active agent.

### BEST MODES FOR CARRYING OUT THE INVENTION

Preferred embodiments for the composition and methods of manufacture are described below.

### EXAMPLE 1

A mixture containing:
(a) 70-90% iron powder (densifier);
(b) 2.5-20% glycerol monostearate (binding agent); and,
(c) 0.5-15% of a blend of glycerol monostearate and polyethylene glycol monostearate (releasing agent);
   was melted and mixed together at a temperature sufficient to melt the binding and releasing agents.

The mixture was then cooled to room temperature and the mass was ground to a fine powder.

A mixture was then formed between 89% and 99% of the above powder and between 0.5% and 10.0% albendazole and between 0.01% and 1% abamectin (the beneficial agents).

This mixture was then pressed into a suitable die at a pressure sufficient to retain the particles together in the shape desired.

This die pressed mixture termed the ‘core’ was then coated. A coating was applied to the external surface of the bolus being formed from a 5-20% (w/v) solution of shellac in absolute alcohol.

After drying, a further impermeable coating was applied to the bolus. This coating is preferably a wax, a mixture of waxes or a wax or waxes combined with an inert material, or a synthetic polymer or similar substance that is substantially impervious to rumen liquor.

### EXAMPLE 2

A composition containing a mixture of 6.5% glycerol monostearate, 6.5% of a blend of glycerol monostearate and polyethylene glycol monostearate and 87% iron powder was melted and mixed at 120-140° C. and subsequently cooled to room temperature.

After cooling, the solid mass was ground to a fine powder with a mechanical grinder.

A mixture containing 96.13% of the above powder, 3.74% albendazole and 0.13% abamectin was mixed and pressed in a die. It was painted with a 13.3% (w/v) solution of shellac in absolute ethanol, and after drying was coated with a mixture of 75% beeswax and 25% carnauba wax.

### EXAMPLE 3

The composition prepared according to Example 2 was administered to a sheep to determine the action of the bolus. The bolus was found to have eroded completely, leaving no residue after approximately 7 days from administration to the sheep.

It should be appreciated from the above examples that there is provided a composition that provides a beneficial agent to an animal in the form of a sustained release vehicle over predetermined time period of days. This has the advantage of supplying the animal with an optimum level of agent during the treatment stage.

Aspects of the present invention have been described by way of example only and it should be appreciated that modifications and additions may be made thereto without departing from the scope thereof as defined in the appended claims.

1. An intra-ruminal composition that releases a therapeutically effective amount of at least one beneficial agent to a ruminant animal over a time period of less than approximately 21 days that includes:
(a) at least one beneficial agent;
(b) at least one densifier material;
(c) at least one binding agent;
(d) at least one releasing agent; and,
wherein the ratio of binding agent to releasing agent ranges from approximately 1 part binding agent to between approximately 0.01 and approximately 2 parts releasing agent to achieve a sustained release rate over a time period of less than approximately 21 days;
and wherein said composition comprises a bolus or pill.

2. The composition as claimed in claim 1 wherein the ratio is approximately 1:1 to achieve release duration of approximately 7 days.

3. The composition as claimed in claim 1 wherein the beneficial agent is at least one anthelmintic compound.

4. The composition as claimed in claim 1 wherein the composition includes two anthelmintic compounds from differing action-families of anti-parasitic activity.

5. (canceled)

6. The composition as claimed in claim 1 wherein the beneficial agent is mineral or nutritional supplements, anti-bacterial agents, anti-viral agents, anti-fungal agents, or other substances beneficial to the health and/or productivity of the ruminant animal.

7. The composition as claimed in claim 1 wherein the beneficial agent is or includes a source of selenium, cobalt or a combination of both of these elements.

8. The composition as claimed in claim 1 wherein the densifier material forms the main component of the central part of the bolus or pill, or the ‘core’, and is characterised by having a density sufficient to retain the bolus or pill within the animal rumen and prevent regurgitation.

9-10. (canceled)

11. The composition as claimed in claim 1 wherein the densifier material is selected from: iron, zinc, tungsten, and cement.

12-13. (canceled)

14. The composition as claimed in claim 1 wherein the binding agent or agents are hydrophobic.

15. The composition as claimed in claim 1 wherein the binding agent or agents are selected from: one or more fats, waxes, gums, fatty acids, fatty acid esters, fatty acid amides, fatty acid alcohols or derivatives thereof, glycerol esters and similar physiologically acceptable organic compounds with equivalent characteristics being that they are physiologically acceptable and are able to bind particulate material.

16. The composition as claimed in claim 1 wherein the releasing agent or agents are characterised by being hydrophilic and having surfactant properties.

17. The composition as claimed in claim 1 wherein the releasing agent or agents are selected from: one or more detergents, soaps, fatty acid salts, polyoxyethylene alcohols and derivatives, polyethylene glycol and derivatives thereof, and like physiologically-acceptable surface-active agents.

18. (canceled)

19. The composition as claimed in claim 1 wherein the bolus or pill erodes away and leaves no residue after the beneficial agent or agents have been released.

20. The composition as claimed in claim 1 wherein as the bolus or pill is eroded in use, beneficial agent or agents are released at a sustained rate during the course of treatment.

21-24. (canceled)

25. The composition as claimed in claim 1 wherein the bolus or pill is coated with a first and second layer of coating agent.

26. (canceled)

27. The composition as claimed in claim 25 wherein the first layer coating is formed from at least one sealing agent, applied to the whole bolus or pill except the distal planar end; and wherein the sealing agent includes natural or synthetic resins.

28-31. (canceled)

32. The composition as claimed in claim 25 wherein the second layer is formed from: a wax, a mixture of waxes or, a wax or waxes combined with an inert material or a synthetic polymer that is substantially impervious to rumen fluid.

33. (canceled)

34. A method of manufacturing an intra-ruminal bolus or pill that releases a therapeutically effective amount of at least one beneficial agent to a ruminant animal over a time period of less than approximately 21 days by the steps of:
(a) forming a substantially homogenous mass by melting and mixing together materials selected from:
i. at least one beneficial agent;
ii. at least one densifier material;
iii. at least one binding agent;
iv. at least one releasing agent; and,
(b) adding the mass to a mould to form a bolus or pill; and,
characterized in that the ratio of binding agent to releasing agent ranges from approximately 1 part binding agent to between approximately 0.01 and approximately 2 parts releasing agent to achieve a sustained release rate over a time period of less than approximately 21 days.

35. A method of manufacturing an intra-ruminal bolus or pill that releases a therapeutically effective amount of at least one beneficial agent to a ruminant animal over a time period of less than approximately 21 days by the steps of:
(a) forming a substantially homogenous mass by melting and mixing together materials selected from:
i. at least one beneficial agent;
ii. at least one densifier material;
iii. at least one binding agent;
iv. at least one releasing agent;
(b) cooling the mixture until it solidifies;
(c) grinding the mixture of step (b) into a fine powder;
(d) forming the ground particles of step (c) into a bolus or pill by compression within a die; and,
characterized in that the ratio of binding agent to releasing agent ranges from approximately 1 part binding agent to between approximately 0.01 and approximately 2 parts releasing agent to achieve a sustained release rate over a time period of less than approximately 21 days.

36. A method of manufacturing an intra-ruminal bolus or pill that releases a therapeutically effective amount of at least one beneficial agent to a ruminant animal over a time period of less than approximately 21 days by the steps of:
(a) forming a substantially homogenous mass by melting and mixing together materials selected from:
i. at least one densifier material;
ii. at least one binding agent;
iii. at least one releasing agent;
(b) cooling the mixture until it solidifies;
(c) grinding the mixture of step (b) into a fine powder;
(d) mixing at least one beneficial agent with the ground particles of step (c);
(e) forming the mixture of step (d) into a bolus or pill by compression within a die; and,
characterized in that the ratio of binding agent to releasing agent ranges from approximately 1 part binding agent to between approximately 0.01 and approximately 2 parts releasing agent to achieve a sustained release rate over a time period of less than approximately 21 days.

37. The method as claimed in claim 34 wherein the mass is added to a mould in step (b) by methods selected from: extrusion, pouring, injection, and combinations thereof.

38-39. (canceled)

40. The method as claimed in claim 34 wherein a further step (c) is completed after step (b) of:
(c) coating the bolus or pill with at least one coating agent.

41. The method as claimed in claim 35 wherein a further step (e) is completed after step (d) of:
(e) coating the bolus or pill with at least one coating agent.

42. The method as claimed in claim 36 wherein a further step (f) is completed after step (e) of:
(f) coating the bolus or pill with at least one coating agent.

43-46. (canceled)

47. The method as claimed in claim 34 wherein the mixture of step (a) is heated to between approximately 120° C. and approximately 140° C.

48. The composition of claim 1, wherein said composition is a bolus.

49. The composition of claim 1, wherein said composition is a pill.
